(19) United States
(12) Patent Application Publication
(10) Pub. No.: US 2025/0228458 A1
(43) Pub. Date: Jul. 17, 2025

(54) PHYSIOLOGICAL SIGNAL MEASUREMENT DEVICE, PHYSIOLOGICAL SIGNAL MEASUREMENT SYSTEM, AND NON-TRANSITORY COMPUTER READABLE STORAGE MEDIUM

(71) Applicant: NIHON KOHDEN CORPORATION, Tokyo (JP)

(72) Inventors: Kota SAEKI, Saitama (JP); Hideaki HIRABARA, Saitama (JP)

(21) Appl. No.: 19/016,499

(22) Filed: Jan. 10, 2025

(30) Foreign Application Priority Data
Jan. 12, 2024 (JP) 2024-003157

(51) Int. Cl. A61B 5/00

(52) U.S. Cl. CPC A61B 5/0053; A61B 5/0082; A61B 5/7221; A61B 5/7264

(57) ABSTRACT

A physiological signal measurement device includes: an information processor of one or more processors configured to calculate, based on change in a detected light intensity that is detected by a light detector configured to detect light emitted from a light emitter and transmitted through a physiological tissue, a capillary refilling time after a compression period in which the physiological tissue is compressed. The information processor uses the detected light intensity to determine whether the change in the detected light intensity is appropriate for calculating the capillary refilling time.

### CROSS-REFERENCE TO RELATED APPLICATIONS

This application is based on and claims priority under 35 USC 119 from Japanese Patent Application No. 2024-003157 filed on Jan. 12, 2024, the contents of which are incorporated herein by reference.

### TECHNICAL FIELD

The presently disclosed subject matter relates to a physiological signal measurement device, a physiological signal measurement system, and a non-transitory computer readable storage medium.

### BACKGROUND ART

As a method of observing a condition of a patient, there is a method of measuring a capillary refilling time. The capillary refilling time is a period of time from compression of a nail bed to recovery of redness after release of compression, and becomes long in a case where there is a problem in circulation of blood. Determination related to the circulation according to the capillary refilling time is also used for triage.

JP5687994B describes a physiological signal measurement device capable of selecting a pulse oximeter mode for calculating a transcutaneous oxygen saturation or the like and a capillary refilling time measurement mode for calculating a capillary refilling time. The physiological signal measurement device is attached to a physiological tissue of a subject. A sensor attachment unit of a tip or the like, which is a physiological tissue of the subject, is provided with a light emitter that emits light, and a light detector that outputs an electric signal corresponding to a detected light intensity of the light.

The light from the light emitter in the above-described physiological signal measurement device is transmitted through a physiological tissue having blood in the capillary and enters the light detector. When the physiological tissue is compressed, blood is removed from the capillary of the physiological tissue, absorption by the blood decreases, and the transmitted light intensity increases. Thereafter, as the compression is released and the blood flows into the physiological tissue of the sensor attachment unit again, absorption by the blood increases and the transmitted light intensity decreases. A time from the release of compression to a time point when the transmitted light intensity becomes a predetermined value is a refilling time.

However, even when the physiological tissue is compressed to measure the capillary refilling time, there may be a case where the blood is not removed without applying sufficient compression to the physiological tissue or a case where a position relation of sensor elements and a compression direction are not aligned. In a change in the transmitted light intensity obtained in this case, there is a possibility that the obtained capillary refilling time becomes inaccurate. Then, it is necessary for a user of the physiological signal measurement device to determine, as viewed from a measurement result or the like, whether measurement on the capillary refilling time is appropriately performed.

### SUMMARY

A physiological signal measurement device according to an embodiment of the presently disclosed subject matter includes an information processor of one or more processors configured to calculate, based on change in a detected light intensity that is detected by a light detector configured to detect light emitted from a light emitter and transmitted through a physiological tissue, a capillary refilling time after a compression period in which the physiological tissue is compressed, in which the information processor uses the detected light intensity to determine whether the change in the detected light intensity is appropriate for calculating the capillary refilling time.

Whether a device is in an attachment state appropriate for calculating a capillary refilling time can be determined without causing a burden on a user of the device.

### BRIEF DESCRIPTION OF DRAWINGS

FIG. 1 is a diagram illustrating a physiological signal measurement system according to an embodiment.

FIG. 2 is a graph of a change in a transmitted light intensity for describing a capillary refilling time.

FIG. 3 is a graph of the change in the transmitted light intensity when compression to a digit is insufficient.

FIG. 4 is Example 1 of calculation on a threshold of a transmitted light intensity change amount due to compression.

FIG. 5 is Example 2 of calculation on the threshold of the transmitted light intensity change amount due to compression.

FIG. 6 is Example 1 of an attachment state in which a position relation between a light emitter and a light detector is deviated.

FIG. 7 is Example 2 of the attachment state in which the position relation between the light emitter and the light detector is deviated.

FIG. 8 is an example of a compression direction in which the position relation between the light emitter and the light detector is deviated.

FIG. 9 is Example 1 of a graph of a change in the transmitted light intensity when the position relation between the light emitter and the light detector is deviated.

FIG. 10 is Example 2 of the graph of the change in the transmitted light intensity when the position relation between the light emitter and the light detector is deviated.

### DESCRIPTION OF EMBODIMENTS

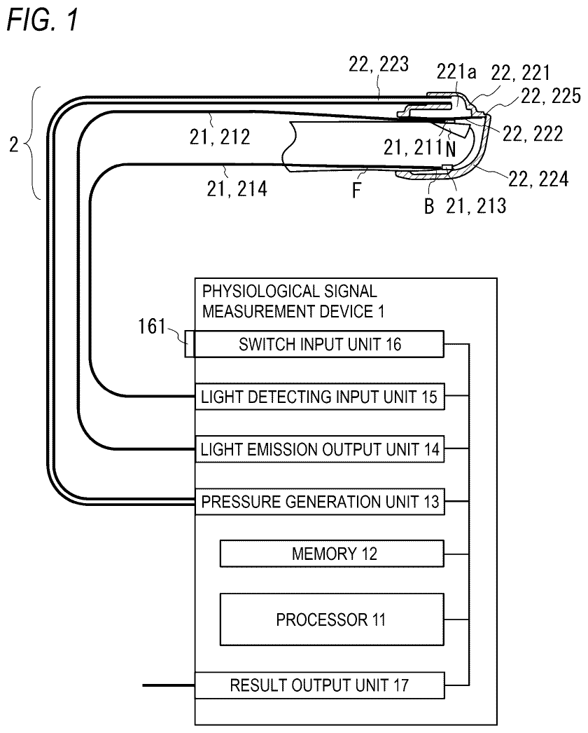

FIG. 1 illustrates a physiological signal measurement system according to an embodiment of the presently disclosed subject matter. The physiological signal measurement system can include a physiological signal obtainer 2 for obtaining a physiological signal, and a physiological signal measurement device 1 that is connected to the physiological signal obtainer 2 and performs measurement or the like on a capillary refilling time CRT.

The physiological signal obtainer 2 according to the embodiment can include a sensor instrument 21 and a support tool 22 for applying compression to a physiological tissue. The sensor instrument 21 can include wirings 212 and 214 respectively connected to a light emitter 211 and a light detector 213. In FIG. 1, the light emitter 211 is attached to a nail N of a digit F, and the light detector 213 is attached to a pad B. The light emitter 211 and the light detector 213 are wound around by a tape (not illustrated) and fixed to the digit F. The support tool 22 covers the light emitter 211 and the light detector 213 from the outside to compress the physiological tissue or release the compression. It is also possible to manually apply compression to the physiological tissue without using the support tool 22.

A cover 221 of the support tool 22 is attached, by a hinge portion 225, to a support body 224 so as to be openable and closable. The support tool 22 is provided with a bag membrane 222 provided on a digit F side of a pressure chamber 221a of the cover 221 to which a tube 223 is connected, and the cover 221 and the bag membrane 222 of the support tool 22 form a bag surrounding the pressure chamber 221a. In addition, the support tool 22 can include the support body 224 from a distal end side of the digit F to a lower side of the pad B. The support body 224 also extends to a side portion of the digit F. The outside of the digit F is surrounded by the support body 224, the bag membrane 222, the cover 221, and the hinge portion 225. FIG. 1 illustrates a cross section of the support tool 22 in an attachment state in which the digit F is inserted into a recess surrounded by the support body 224, the bag membrane 222, and the like. The digit F, which is a physiological tissue, is compressed from the outside by the support tool 22.

On the other hand, the physiological signal measurement device 1 can include one or more processors 11, one or more memories 12, a pressure generation unit 13, a light emission output unit 14, a light detecting input unit 15, a switch input unit 16, and a result output unit 17. The pressure generation unit 13 generates an air pressure and supplies the air pressure to the tube 223. The light emission output unit 14 causes the light emitter 211 to emit light by supplying a current to the wiring 212. The light emitted from the light emitter 211 is transmitted through the physiological tissue and is detected by the light detector 213. When the light passes through the physiological tissue, light absorption or reflection occurs in the physiological tissue, and a part of the finally transmitted light reaches the light detector 213. Then, a voltage corresponding to a detected light amount detected by the light detector 213 is detected by the light detecting input unit 15, converted into a digital signal, and sent to the processor 11. The switch input unit 16 can include an inspection switch 161, and in a case where the inspection switch 161 is operated, the capillary refilling time CRT in the physiological signal measurement device 1 is calculated. The inspection switch 161 in the embodiment is a physical switch, but may be a button displayed on a display device having an input function by contact. The processor 11 calculates, based on change in the detected light intensity detected by the light detector 213, the capillary refilling time CRT after a compression period in which the physiological tissue is compressed from the outside. In a case where blood circulation abnormality is determined based on a result of the calculation, circulation abnormality information is output from the result output unit 17 and is notified to a user by a sound generation device, a display device, or the like (not illustrated). Control of the physiological signal measurement device 1 is performed by the processor 11 which is an information processor.

At the time of evaluating a blood circulation state, the air generated by the pressure generation unit 13 of the physiological signal measurement device 1 is supplied to the pressure chamber 221a of the support tool 22 via the tube 223. The pressure generation unit 13 and the processor 11 that controls the pressure generation unit 13 functions as an automatic compression device that automatically performs compression and forms a compression period. An inflation target value and a compression time are stored in the memory 12, and a pressure of the pressure chamber 221a is adjusted by the air pressure generated from the pressure generation unit 13 due to the stored inflation target value and compression time. As the air pressure in the pressure chamber 221a in the support tool 22 increases, the bag membrane 222 expands in a direction of the nail N to compress the digit F from the outside. A blood amount in the capillary of the digit F, which is a physiological tissue, is reduced by compression. The light used in the sensor instrument 21 uses a wavelength with high absorptance due to blood. Therefore, in a case where the blood amount of the physiological tissue through which light passes decreases, the absorption of light due to the blood decreases, and the transmitted light intensity I increases. The transmitted light intensity I is the detected light intensity, which is the light intensity obtained by the light detector 213, of the light emitted from the light emitter 211 and transmitted or reflected by the physiological tissue, and changes depending on increase or decrease in the blood amount in the physiological tissue.

After a predetermined compression period elapses, the pressure is released by the pressure generation unit 13 of the physiological signal measurement device 1. Thus, expansion of the bag membrane 222 in the direction of the nail N is eliminated, and the digit F is released from the compression. Accordingly, the blood returns to the capillary of the physiological tissue, the absorption of light due to the blood increases, and the transmitted light intensity I decreases. In the physiological signal measurement device 1, the transmitted light intensity I is digitized by the light detecting input unit 15, and the capillary refilling time CRT in which the transmitted light intensity I is smaller than a predetermined value Ir is calculated by the processor 11. In a case where the capillary refilling time CRT is equal to or longer than the predetermined time, the circulation abnormality information indicating the blood circulation abnormality is transmitted from the result output unit 17, and the blood circulation abnormality is notified.

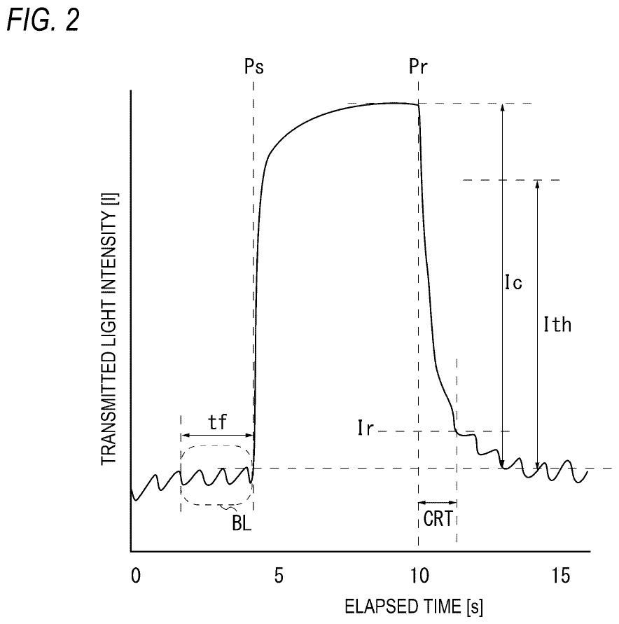

The measurement on the capillary refilling time CRT will be described using a graph of a change in a transmitted light intensity which is a change in the transmitted light intensity I in FIG. 2. A vertical axis in FIG. 2 indicates the transmitted light intensity I which is the detected light intensity indicating the detected light amount of the light detector 213 and being the light intensity after the light emitted by the light emitter 211 passes through the physiological tissue of the digit F. A horizontal axis represents elapsed time. The compression on the digit F starts at a compression start time Ps, and is released at a compression release time Pr. Before the compression start time Ps of the digit F, the transmitted light intensity I is small because the blood amount of the physiological tissue is large. In this period, pulsation is observed in the transmitted light intensity I.

At the compression start time Ps, in a case where the air pressure in the pressure chamber 221a is increased by the air sent from the physiological signal measurement device 1 and the digit F is pressed by the support tool 22, the blood amount in the physiological tissue decreases and the absorption of light decreases. Therefore, the transmitted light intensity I detected by the light detector 213 increases. In FIG. 2, compression is started at the compression start time Ps, and compression is released at the compression release time Pr after about six seconds elapse. A compression period Ps-Pr is from the compression start time Ps to the compression release time Pr. In the compression period Ps-Pr, there is almost no pulsation of the transmitted light intensity I, unlike before the compression start time Ps.

After the compression release time Pr, the transmitted light intensity I decreases, and the transmitted light intensity I at the same level as the compression start time Ps is obtained. In the embodiment, a time at which the transmitted light intensity I decreases to the predetermined value Ir after the compression release time Pr is referred to as the capillary refilling time CRT. The fact that the transmitted light intensity I decreases to the predetermined value Ir indicates that the redness of the physiological tissue of the digit F is recovered. Then, in a case where the capillary refilling time CRT is equal to or longer than a predetermined number of seconds, the circulation abnormality information is transmitted from the result output unit 17, and the blood circulation abnormality is notified. The predetermined value Ir may be stored in the memory 12 of the physiological signal measurement device 1 as a fixed value set in advance, or may be calculated using the transmitted light intensity I before the compression start time Ps or immediately before the compression release time Pr. In addition, the predetermined value Ir may be calculated using variation in the transmitted light intensity I due to compression, for example, the predetermined value Ir can be a value obtained by adding, to the transmitted light intensity I at the compression start time Ps, a predetermined rate (for example, 10%) of a transmitted light intensity change amount Ic, which is a difference between the transmitted light intensity I at the compression start time Ps and the transmitted light intensity I at the compression release time Pr. In addition, the predetermined value Ir may be a value at which a rate of change in the transmitted light intensity I after the compression release time Pr is equal to or less than a certain value.

However, even when the physiological tissue is compressed to calculate the capillary refilling time CRT, there may be a case where the blood is not removed without applying sufficient compression to the physiological tissue or a case where the position relation between the light emitter 211 and the light detector 213 and the compression direction are not aligned. In the change in the transmitted light intensity I obtained in this case, there is a possibility that the obtained capillary refilling time CRT becomes inaccurate.

### When Compression is Insufficient

When the compression on the digit F is insufficient since the digit F is small or the cover 221 of the support tool 22 is not fixed in a closed state, it is impossible to detect an abnormality of blood circulation by the capillary refilling time CRT. When either of the following two features has a change in the transmitted light intensity I, there is a possibility that the compression on the physiological tissue is insufficient. (1) Pulsation is observed in the compression period Ps-Pr from the compression start time Ps to the compression release time Pr.
(2) The transmitted light intensity change amount Ic, which is the change amount in the transmitted light intensity I due to compression, is small.

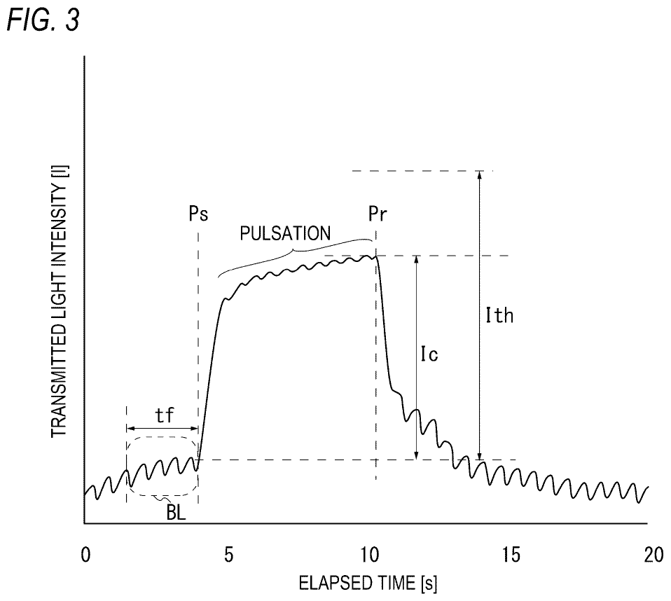

FIG. 3 illustrates a graph of the change in the transmitted light intensity which is a change in the transmitted light intensity I when the compression on the digit F is insufficient. FIG. 2 illustrates a change in the transmitted light intensity I when the compression is sufficient. Determination of insufficient compression will be described with reference to FIGS. 2 and 3.

### (1) Determination on Insufficient Compression by Remaining of Pulsation

When pulsation is observed in the compression period Ps-Pr, it is determined that the compression is insufficient. Specifically, a waveform of the transmitted light intensity I during the compression period Ps-Pr from the compression start time Ps to the compression release time Pr illustrated in FIGS. 2 and 3 is passed through a band-pass filter, and in a case where an output is equal to or more than a predetermined value, it is determined that the change in the detected light intensity is inappropriate for calculating the capillary refilling time CRT. A passband of the band-pass filter is set to a frequency that may be taken by the heartbeat. Since no pulsation of a frequency component of the heartbeat is observed in the waveform of the transmitted light intensity I during the compression period Ps-Pr in FIG. 2, the output does not exceed a predetermined value and no pulsation is detected. On the other hand, since pulsation of the frequency component of the heartbeat is observed in the waveform of the transmitted light intensity I in the compression period Ps-Pr of FIG. 3, the output is equal to or more than the predetermined value, so that the pulsation is detected, and it is determined that the compression is insufficient.

The band-pass filter according to the present embodiment is provided as software in the physiological signal measurement device 1, and calculates an output by the processor 11. However, a circuit may be provided in a part of the light detecting input unit 15 or the like. In addition, a high-pass filter may be used instead of the band-pass filter. A determination waveform of the transmitted light intensity I used for the determination may be a waveform after a predetermined time elapses from the compression start time Ps. For example, in the waveforms illustrated in FIGS. 2 and 3, a determination waveform starting one second after the compression start time Ps and ending at the compression release time Pr can be used except for a period in which the transmitted light intensity I immediately after the compression start time Ps greatly changes. At the time of determining whether pulsation is observed in the compression period Ps-Pr, other frequency analysis may be used in addition to the above-described filter. In addition, image recognition of a waveform, feature recognition of a waveform (template matching or the like set in advance), AI recognition, or the like can be used.

(2) Determination on Insufficient Compression according to Transmitted Light Intensity Change Amount Ic of Transmitted Light Intensity I

As illustrated in FIGS. 2 and 3, the transmitted light intensity change amount Ic according to the present embodiment is obtained as a difference between the transmitted light intensity I at the compression start time Ps and the transmitted light intensity I at the compression release time Pr. When the transmitted light intensity change amount Ic is smaller than a determination threshold Ith, it is determined that the change in the detected light intensity is inappropriate for calculating the capillary refilling time CRT. In the change in the transmitted light intensity illustrated in FIG. 2, the transmitted light intensity change amount Ic>the determination threshold Ith, and thus it is determined that the change in the detected light intensity is appropriate for calculating the capillary refilling time CRT. On the other hand, in the change in the transmitted light intensity illustrated in FIG. 3, the transmitted light intensity change amount Ic<the determination threshold Ith, and thus it is determined that the change in the detected light intensity is inappropriate for calculating the capillary refilling time CRT.

The transmitted light intensity change amount Ic according to the present embodiment is a difference between the transmitted light intensity I at the compression start time Ps and the transmitted light intensity I at the compression release time Pr. However, the transmitted light intensity change amount Ic may be another amount such as a difference between the transmitted light intensity I at the compression release time Pr and an average value of the transmitted light intensity I of a base line BL in a pre-start predetermined period tf which is a period immediately before the compression start time Ps. For example, the transmitted light intensity change amount Ic may be a difference between the predetermined value Ir for calculating the above-described capillary refilling time CRT and the transmitted light intensity I at the compression release time Pr. In addition, the transmitted light intensity change amount Ic may be a difference between an average value of the transmitted light intensity I in a predetermined period after the variation of the transmitted light intensity I becomes equal to or less than a certain value after the compression release time Pr and the transmitted light intensity I at the compression release time Pr.

How the determination threshold Ith is set includes the following two examples.

### (2a) Determination Threshold Ith as Fixed Value

The determination threshold Ith is a fixed value set in advance. The determination threshold Ith is stored, as a fixed value, in the memory 12 of the physiological signal measurement device 1.

(2b) Determination Threshold Ith being Calculated by Waveform (Base Line BL) Before Compression Start Time Ps

A value calculated by using at least one of a pulsation rate P of the waveform (base line BL) before the compression start time Ps, the transmitted light intensity I, and a light emitter emission intensity L is set as the determination threshold Ith. In FIGS. 2 and 3, the waveform of the pre-start predetermined period tf (about 2.5 seconds) immediately before the compression start time Ps is defined as the base line BL. In the calculation, data of the base line BL of the transmitted light intensity I measured on a large number of subjects is used. Here, “Pulsation rate” is calculated from pulsating components of signal and is different from “Pulse Rate”. The “pulsation rate” as expressed in this disclosure is an index that indicates a magnitude of pulse signal, and is used to evaluate a state of blood flow. In terms of terminology, “Pulsation rate” is also expressed as “Pulse-amplitude Index” or “Perfusion Index (PI)”.

(2b1) Determination on Determination Threshold Ith Based on Function Obtained from Data on Pulsation Rate P Before Compression and Transmitted Light Intensity Change Amount Ic Due to Compression

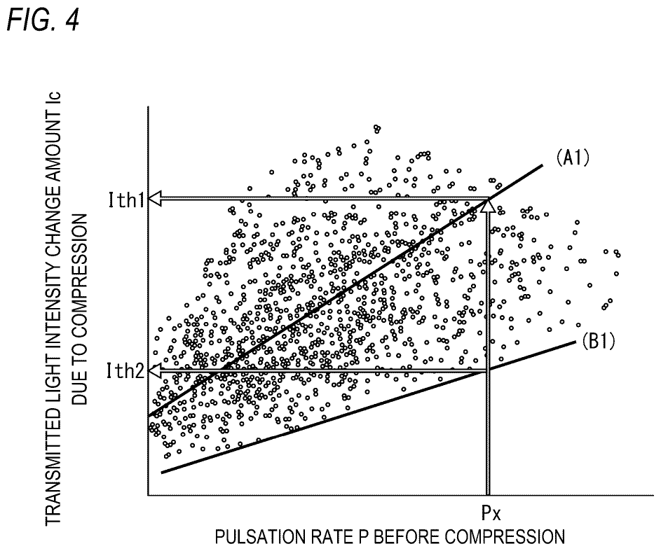

FIG. 4 is a graph in which, based on data on the change in the transmitted light intensity which is a change in the transmitted light intensity I measured on a large number of subjects, the pulsation rate P before compression is plotted on a horizontal axis, and the transmitted light intensity change amount Ic due to compression is plotted on a vertical axis. In the present embodiment, the pulsation rate P is obtained by (maximum value−minimum value)/average value of the transmitted light intensity I in the pre-start predetermined period tf.

Points in FIG. 4 are distributed from the lower left to the upper right as a whole, and it can be seen that the pulsation rate P of the base line BL in the pre-start predetermined period tf and the transmitted light intensity change amount Ic have a certain degree of correlation. This correlation is considered to be because, in a case where the blood amount in a measurement unit is large, the pulsation rate P is large, and the blood amount excluded due to compression is large, so that the transmitted light intensity change amount Ic also increases. (A1) is a regression line of this data. Then, the determination threshold Ith can be obtained from the pulsation rate P before compression by using the regression line (A1) which is a linear function. For example, as illustrated in FIG. 4, when the pulsation rate P is Px, Ith1, which is a value of a Y axis of the regression line (A1) corresponding to Px, is set as the determination threshold Ith.

(B1) is a lower limit line indicating a lower limit of distribution of the points. Then, the determination threshold Ith can be obtained from the pulsation rate P before compression by using the lower limit line (B1) which is a linear function. For example, as illustrated in FIG. 4, when the pulsation rate P is Px, Ith2, which is a value of the Y axis of the lower limit line (B1) corresponding to Px, is set as the determination threshold Ith. The lower limit may be not only a line which is a linear function, but also a function of a polygonal line or a curve.

(2b2) Determination on Determination Threshold Ith based on Function Obtained from Value of Regression Equation Obtained by Multiple Regression Analysis and Data on Transmitted Light Intensity Change Amount Ic

As can be seen from the fact that the transmitted light intensity change amount Ic and the pulsation rate P before compression in FIG. 4 are distributed from the lower left to the upper right, they have some correlation, but it is desirable to determine the determination threshold value Ith by an index having a greater correlation. Therefore, multiple regression analysis may be performed, based on the data on the change in the transmitted light intensity measured on a large number of subjects, using “the transmitted light intensity change amount due to compression Ic” as an objective variable and “the pulsation rate P before compression, the transmitted light intensity I, the light emitter emission intensity L”, and the like as explanatory variables, and the determination threshold Ith may be calculated from the regression equation at that time.

For example, since the transmitted light intensity I varies in a case where the thickness of the digit F varies, the pulsation rate P before compression may also vary depending on the thickness of the digit F. Here, it is considered that the transmitted light intensity change amount Ic is set as an objective variable with the pulsation rate P before the compression and a transmitted light intensity DC value Id, which is a DC component of the transmitted light intensity I before compression, as explanatory variables. In this case, as follows.

The transmitted light intensity change amount Ic is a linear function of the pulsation rate P and the transmitted light intensity DC value Id.

$$[ Equation 1 ]$$

$$Transmitted light intensity change amount Ic = a ⋆ pulsation rate P + b ⋆ transmitted light intensity DC value Id + c$$

a, b, and c are constants.

Then, multiple regression analysis is performed using the transmitted light intensity change amount Ic as an objective variable, the pulsation rate P and the transmitted light intensity DC value Id as explanatory variables to determine the constants a, b, and c.

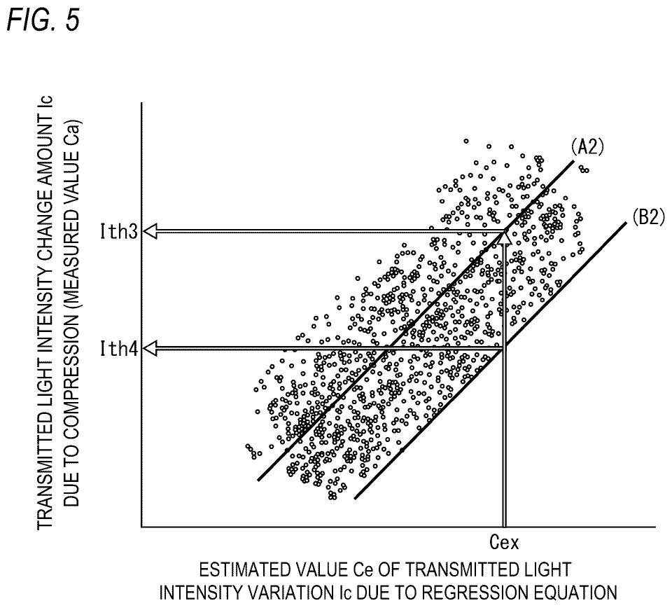

Thus, an estimation value Ce of the transmitted light intensity change amount Ic according to the regression equation can be calculated by determining the pulsation rate P and the transmitted light intensity DC value Id. In FIG. 5, multiple regression analysis is performed to define the constants a, b, and c, and the estimation value Ce calculated as follows is taken as a horizontal axis. A vertical axis is a measured value Ca of the transmitted light intensity change amount Ic due to compression.

$$Estimation value Ce = a ⋆ pulsation rate P + b ⋆ transmitted light intensity DC value Id + c \qquad [ Equation 2 ]$$

As in the example of FIG. 4, the pulsation rate P is (maximum value−minimum value)/average value of the transmitted light intensity I in the pre-start predetermined period tf.

FIG. 5 is a graph in which the estimation value Ce calculated by Equation [2] and the transmitted light intensity change amount Ic due to the compression are plotted by the data on the change in the transmitted light intensity which is a change in the transmitted light intensity I measured on a large number of subjects. It is seen that in FIG. 5, the distribution from the lower left to the upper right is stronger than that in FIG. 4, and the correlation is large.

(A2) is a regression line obtained by points in FIG. 5. The determination threshold Ith can be obtained from the estimation value Ce by using the regression line (A2) which is a linear function. For example, as illustrated in FIG. 5, when the estimation value Ce is Cex, Ith3, which is a value of a Y axis of the regression line (A2) corresponding to Cex, is set as the determination threshold Ith.

(B2) is a lower limit line indicating a lower limit of the distribution of the points. The determination threshold Ith can be obtained from the estimation value Ce by using the lower limit line (B2) which is a linear function. For example, as illustrated in FIG. 5, when the estimation value Ce is Cex, Ith4, which is a value of the Y axis of the lower limit line (B2) corresponding to Cex, is set as the determination threshold Ith. The lower limit may be not only a line which is a linear function, but also a function of a polygonal line or a curve.

In the present embodiment, in a case where the state of (1) or (2) is detected and it is determined that compression is insufficient, re-measurement by increasing the inflation target value and compressing the physiological tissue or re-measurement by increasing the compression time and compressing the physiological tissue is automatically performed. In addition, a notification may be performed such that re-measurement by increasing the inflation target value is prompted or re-measurement by increasing the compression time is prompted. The notification can also be used when compression is manually performed. When it is determined that the compression is insufficient even if the re-measurement is performed, the notification that the measurement is impossible may be performed. In addition, when the state of (1) or (2) is detected, only a notification that the capillary refilling time CRT is not measurable may be performed. In the present embodiment, regarding the notification, information corresponding to the notification is sent from the result output unit 17 and the notification is performed from a sound generation device, a display device, or the like (not illustrated).

In the embodiment, the data on the change in the transmitted light intensity I measured on a large number of subjects is used in FIGS. 4 and 5. However, a plurality of pieces of data on the change in the transmitted light intensity I measured on one subject may be used. The data on the change in the transmitted light intensity measured on at least one subject can be used.

Positional Deviation Between Light Emitter 211 and Light Detector 213, and, Deviation in Compression Direction

As described above, not only a case where the compression on the digit F which is the physiological tissue is insufficient, but also a case where the position relation between the light emitter 211 and the light detector 213 which are attached to the digit F and the compression direction to the digit F are inappropriate are inappropriate for calculating the capillary refilling time CRT. When either of the following two features has a change in the transmitted light intensity I, there is a possibility that the position relation between the light emitter 211 and the light detector 213 and the compression direction to the digit F is inappropriate. (3) Case where a protrusion shape occurs in at least one of immediately after the compression start time Ps and immediately after the compression release time Pr. (4) Case where the decrease in the transmitted light intensity I from the base line BL before a compression section occurs.

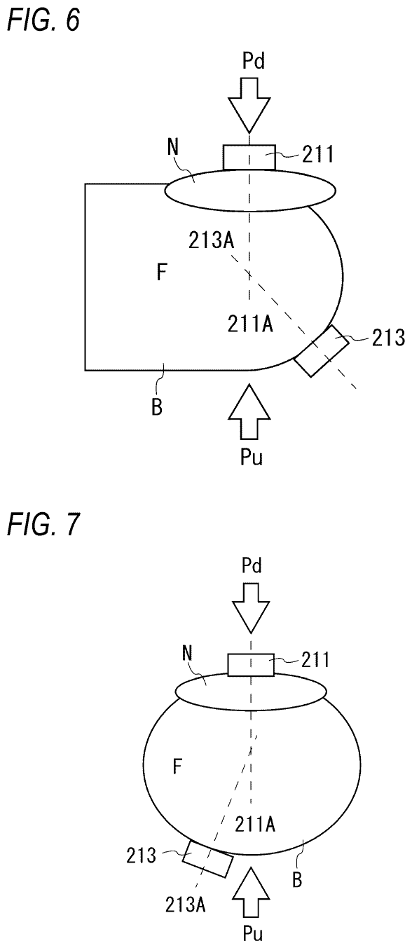

A specific example when the position relation between the light emitter 211 and the light detector 213 and the compression direction are not aligned will be described below. FIGS. 6 and 7 illustrate Example 1 and Example 2 illustrating attachment states in which the position relation between the light emitter 211 and the light detector 213 is deviated. A pressure from the bag membrane 222 attached to the cover 221 of the support tool 22 compresses the digit F from above as downward compression Pd. In addition, since the digit F is pressed by the support body 224, the digit F is compressed from below as upward compression Pu from the support body 224. FIG. 6 is a side view of the digit F, and in the attachment state of FIG. 6, the light detector 213 is attached to the distal end side of the digit F with respect to the pad B. Therefore, a light emitting optical axis 211A, which is an optical axis of the light emitter 211 installed on the nail N, and a light detecting optical axis 213A, which is an optical axis of the light detector 213, deviate from each other and intersect at a predetermined angle or more. In Example 1 of FIG. 6, the upward compression Pu and the downward compression Pd substantially coincide with each other.

FIG. 7 is a view of the attachment state of the light emitter 211 and the light detector 213 of Example 2, which is another example from FIG. 6, as viewed from a tip side of the digit F. In the attachment state of FIG. 7, the light detector 213 is attached to a side surface side of the digit F with respect to the pad B. Therefore, the light emitting optical axis 211A, which is the optical axis of the light emitter 211 installed on the nail N, and the light detecting optical axis 213A, which is the optical axis of the light detector 213, deviate from each other and intersect at a predetermined angle or more. In the attachment state of Example 2 illustrated in FIG. 7, directions of the upward compression Pu and the downward compression Pd substantially coincide with each other. Although not illustrated in the drawings, the position relation between the light emitter 211 and the light detector 213 is inappropriate as in a case where the light detector 213 is located obliquely to the distal end side of the digit F or a case where the light emitting optical axis 211A and the light detecting optical axis 213A are twisted.

In a case where the light emitting optical axis 211A and the light detecting optical axis 213A intersect with each other at an angle equal to or more than a predetermined angle or are twisted as in the attachment state of Examples 1 and 2 illustrated in FIGS. 6 and 7, the light emitted from the light emitter 211 is less likely to enter the light detector 213, which is inappropriate for calculating the capillary refilling time CRT. Such an attachment state of the light emitter 211 and the light detector 213 occurs when fixing of the light emitter 211 and the light detector 213 to the digit F by a tape (not illustrated) is inappropriate.

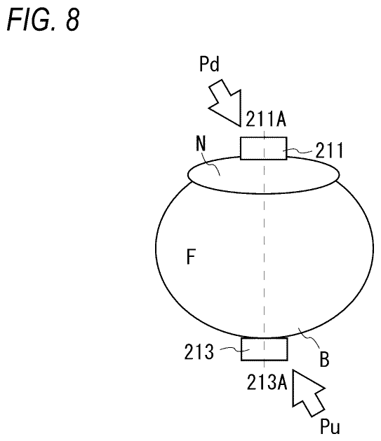

FIG. 8 illustrates an example in which the directions of the upward compression Pu and the downward compression Pd are deviated from each other. In this attachment state, the light emitting optical axis 211A which is the optical axis of the light emitter 211 installed on the nail N and the light detecting optical axis 213A which is the optical axis of the light detector 213 do not deviate from each other and coincide with each other. However, in a case where the digit F is compressed in such a compression state, the positions of the light emitter 211 and the light detector 213 are deviated in a horizontal direction by the upward compression Pu and the downward compression Pd whose directions are deviated, and the light emitting optical axis 211A and the light detecting optical axis 213A are deviated. Therefore, the light emitted from the light emitter 211 is less likely to enter the light detector 213, which is inappropriate for calculating the capillary refilling time CRT. For example, when attachment of the support tool 22 to the digit F is poor or the size of the digit F and the support tool 22 is not suitable, deviation in the compression direction occurs as illustrated in FIG. 8.

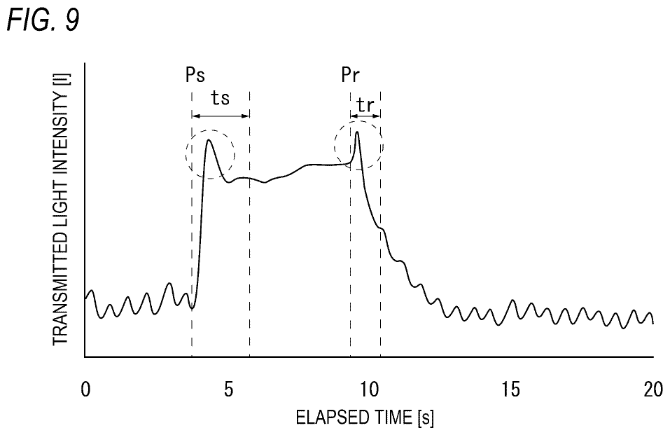

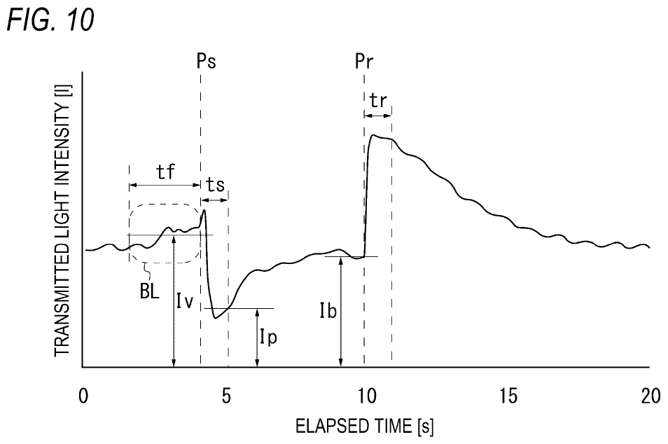

A waveform having a special shape occurs in the attachment state in which the position relation between the light emitter 211 and the light detector 213 is deviated as in Examples 1 and 2 of FIGS. 6 and 7 or in the compression state in which the directions of the upward compression Pu and the downward compression Pd are deviated as in FIG. 8. The same applies to the attachment state in which both the position relation between the light emitter 211 and the light detector 213 and the directions of the upward compression Pu and the downward compression Pd are deviated. From this waveform, it is possible to determine whether to be appropriate for calculating the capillary refilling time CRT. The graphs of the change in the transmitted light intensity I illustrated in FIGS. 9 and 10 as Examples 1 and 2 indicate that the above-described deviation occurs.

The transmitted light intensity I in FIG. 9 in Example 1 indicates a change of increasing immediately after the compression start time Ps, but slightly decreasing after some time passes, and once increasing immediately after the compression release time Pr and then greatly decreasing. As described above, in a case where the digit F is compressed, the blood of the physiological tissue is removed, and absorption due to the blood is reduced, so that the transmitted light intensity I increases. However, in a case where the position relation between the light emitter 211 and the light detector 213 is deviated due to compression immediately thereafter, the angle of the light detecting optical axis 213A illustrated in FIGS. 6 and 7 is changed, and the light emitter 211 and the light detector 213 is deviated in a direction such that the angle between the light emitting optical axis 211A and the light detecting optical axis 213A is close to 90°. As in the attachment states of Examples 1 and 2 illustrated in FIGS. 6 and 7, in a case where the position relation between the light emitter 211 and the light detector 213 is deviated before compression, the deviation is enlarged due to the compression, and the transmitted light reaching the light detector 213 decreases. In addition, even when the position relation between the light emitter 211 and the light detector 213 is not deviated, in a case where the directions of the upward compression Pu and the downward compression Pd are deviated as illustrated in FIG. 8, the position relation between the light emitter 211 and the light detector 213 is deviated due to compression. Accordingly, the light emitting optical axis 211A and the light detecting optical axis 213A are also deviated, and the transmitted light reaching the light detector 213 decreases. A protrusion shape in the compression period Ps-Pr as illustrated in the drawing occurs due to the influence of the decrease in the transmitted light against the increase in the transmitted light of the transmitted light intensity I accompanying the decrease of blood at the time of compression.

In a case where the influence of the decrease in the transmitted light is further increased, the detected light intensity in the compression period Ps-Pr is smaller than the detected light intensity before the compression period Ps-Pr, and is as illustrated in FIG. 10 as Example 2. In FIG. 10, immediately after the compression start time Ps, the transmitted light intensity I is lower than the base line BL before the compression start time Ps.

Specifically, positional deviation of a sensor and deviation in the compression direction are determined in the following cases illustrated in FIG. 9. (3) Presence or Absence of Protrusion Shape Immediately After Compression Start Time Ps and Immediately After Compression Release Time Pr

The processor 11, which is an information processor, determines that the change of the detected light intensity is inappropriate when a peak of the transmitted light intensity I is present, in the change in the detected light intensity, in at least one of a pre-start predetermined period ts which is a predetermined period immediately after the compression start time Ps and a predetermined period after release tr which is a predetermined period immediately after the compression release time Pr. The presence of the peak immediately after the compression start time Ps and immediately after the compression release time Pr can be detected from a change over time in a differential value of the transmitted light intensity I in the predetermined period after start ts or the predetermined period after release tr. In addition, it is also possible to detect, by another method, whether a maximum value is present in the predetermined period after start ts or the predetermined period after release tr.

Further, the processor 11, which is an information processor, may determine that the change of the detected light intensity is inappropriate when a peak of the transmitted light intensity I is present, in the change of the detected light intensity, in one of immediately after the compression start time Ps and immediately after the compression release time Pr and the transmitted light intensity I in the vicinity of the peak of the transmitted light intensity I (within predetermined range from peak value) is present in the other of immediately after the compression start time Ps and immediately after the compression release time Pr.

Specifically, it is also possible to determine positional deviation of a sensor and deviation in the compression direction in the following case illustrated in FIG. 10. (4) Decrease in Transmitted Light Intensity I from Base Line BL Before Compression Section

In FIG. 10, the waveform in the pre-start predetermined period tf (about 2.5 seconds) immediately before the compression start time Ps is defined as the base line BL. The positional deviation of the sensor or the deviation in the compression direction is determined when a value of a transmitted light intensity at the time of compression Ip, which is the transmitted light intensity I after the predetermined period after start ts (1 second) from the compression start time Ps, is smaller than a pre-compression transmitted light average Iv, which is an average of the transmitted light intensity I at the base line BL.

Regarding the change in the transmitted light intensity of the shape in FIG. 10, it is possible to determine that the positional deviation of the sensor or the deviation in the compression direction is present when an average value of the transmitted light intensity I in the predetermined period after release tr from the compression release time Pr is larger than a transmitted light intensity at the time of release Ib which is the transmitted light intensity I at the compression release time Pr. In addition, it is possible to determine by another method such as detection from a change over time in the differential value of the transmitted light intensity I immediately after the compression start time Ps or immediately after the compression release time Pr.

In the present embodiment, in a case where the state of the above (3) or (4) is detected and it is determined that the change in the detected light intensity is inappropriate for calculating the capillary refilling time CRT, position direction inappropriateness information is transmitted from the result output unit 17, and a notification that the position relation between the light emitter 211 and the light detector 213 and the compression direction are inappropriate is performed from the sound generation device, the display device (not illustrated), or the like. In addition, a notification of prompting correction on the position of the sensor or correction on the compression direction may be performed. Both the notification of inappropriateness and the notification of prompting the correction may be performed, or either of the notification of inappropriateness and the notification of prompting the correction may be performed. A user who receives the notification can calculate the capillary refilling time CRT by reattaching the light emitter 211 and the light detector 213 to the digit F or reattaching the support tool 22 to the digit F. The notification can also be used when compression is manually performed. The user who receives the notification can calculate the capillary refilling time CRT by reattaching the light emitter 211 and the light detector 213 to the digit F or compressing the digit F paying attention to the compression direction. In addition, when the state of (3) or (4) is detected, only the notification that the capillary refilling time CRT is not measurable may be performed.

The compression start time Ps and the compression release time Pr in the embodiment can be determined based on control from the processor 11 to the pressure generation unit 13 in the physiological signal measurement device 1. However, the compression start time Ps and the compression release time Pr may be detected from the waveform of the transmitted light intensity I.

In the embodiment, the compression period Ps-Pr is automatically formed, but the user may manually form a compression state resulting in the compression period Ps-Pr by, for example, pressing the digit F of the subject. In this case, the compression start time Ps and the compression release time Pr can be detected from the waveform of the transmitted light intensity I.

In the embodiment, the circulation abnormality information, the position direction inappropriateness information, and the like are output from the result output unit 17, and are notified from the sound generation device, the display device, or the like. However, a notification unit of the sound generation device, the display device, or the like may be provided in the physiological signal measurement device 1 to perform a notification from the notification unit. The physiological signal measurement device may use one of various determinations of being inappropriate for calculating the above-described capillary refilling time CRT, and may use a combination of various determinations.

The processor 11 of the physiological signal measurement device 1 in the above-described embodiment is a computer, and performs a physiological signal measurement method including a capillary refilling time calculation procedure of calculating, based on change in a detected light intensity that is detected by the light detector 213 configured to detect light emitted from the light emitter 211 and transmitted through a physiological tissue, the capillary refilling time CRT after the compression period Ps-Pr in which the physiological tissue is compressed, and a calculation validity determination step of using the detected light intensity to determine whether the change in the detected light intensity is appropriate for calculating the capillary refilling time CRT.

In addition, the processor 11 of the physiological signal measurement device 1 is a computer, and implements, by a physiological signal measurement program, a capillary refilling time calculation function of calculating, based on change in a detected light intensity that is detected by the light detector 213 configured to detect light emitted from the light emitter 211 and transmitted through a physiological tissue, the capillary refilling time CRT after the compression period Ps-Pr in which the physiological tissue is compressed, and a calculation validity determination function of using the detected light intensity to determine whether the change in the detected light intensity is appropriate for calculating the capillary refilling time CRT.

A physiological signal measurement program can be stored in a computer-readable storage medium, the program causing a computer to implement a capillary refilling time calculation procedure of calculating, based on change in a detected light intensity that is detected by the light detector 213 configured to detect light emitted from the light emitter 211 and transmitted through a physiological tissue, the capillary refilling time CRT after the compression period Ps-Pr in which the physiological tissue is compressed, and a calculation validity determination step of using the detected light intensity to determine whether the change in the detected light intensity is appropriate for calculating the capillary refilling time CRT. The storage medium can include a transitory storage medium and a non-transitory storage medium.

Although the embodiment and the like of the presently disclosed subject matter is described in detail above, specific configurations are not limited to the embodiment and the like, and changes in design and the like without departing from the gist of the presently disclosed subject matter are also included in the presently disclosed subject matter. The above-described embodiment and the like can be combined using techniques of one another as long as without particular contradictions or problems in the object, configurations, and the like.

The one or more memories can include, for example, a read only memory (ROM) that stores various computer programs and the like, and a random access memory (RAM) having a plurality of work areas in which various computer programs to be executed by the processor are stored. The one or more processors is, for example, a central processing unit (CPU), which loads a specified computer program from the various computer programs incorporated in the ROM onto the RAM and executes various processes in cooperation with the RAM.

1. A physiological signal measurement device comprising:
an information processor of one or more processors configured to calculate, based on change in a detected light intensity that is detected by a light detector configured to detect light emitted from a light emitter and transmitted through a physiological tissue, a capillary refilling time after a compression period in which the physiological tissue is compressed, wherein
the information processor uses the detected light intensity to determine whether the change in the detected light intensity is appropriate for calculating the capillary refilling time.

2. The physiological signal measurement device according to claim 1, wherein
in a case where pulsation is detected in the detected light intensity in the compression period, the information processor determines that the change in the detected light intensity is inappropriate for calculating the capillary refilling time.

3. The physiological signal measurement device according to claim 1, wherein
in a case where a transmitted light intensity change amount, which is an amount of change in a transmitted light intensity due to compression, is smaller than a determination threshold, the information processor determines that the change in the detected light intensity is inappropriate for calculating the capillary refilling time.

4. The physiological signal measurement device according to claim 3, wherein
the determination threshold is a fixed value set in advance.

5. The physiological signal measurement device according to claim 3, wherein
the determination threshold is a value calculated using at least one of the detected light intensity before the compression period and a pulsation rate calculated from the fluctuation in the detected light intensity before the compression period.

6. The physiological signal measurement device according to claim 5, wherein
the determination threshold is calculated based on a distribution of data on a change in a transmitted light intensity measured on at least one subject.

7. The physiological signal measurement device according to claim 6, wherein
the determination threshold is calculated based on a regression line or a lower limit of the distribution of the data on the change in the transmitted light intensity measured on at least one subject.

8. The physiological signal measurement device according to claim 1, wherein
the information processor determines that the change in the detected light intensity is inappropriate when in the change in the detected light intensity, a peak of the transmitted light intensity is present in one of immediately after a compression start time and immediately after a compression release time.

9. The physiological signal measurement device according to claim 1, wherein
the information processor determines that the change in the detected light intensity is inappropriate when in the change in the detected light intensity, a peak of the transmitted light intensity is present in one of immediately after the compression start time and immediately after the compression release time and a transmitted light intensity within a predetermined range from a value of the peak is present in the other of immediately after the compression start time and immediately after the compression release time.

10. The physiological signal measurement device according to claim 1, wherein
the information processor determines that the change in the detected light intensity is inappropriate in a case where the detected light intensity in the compression period is smaller than the detected light intensity before the compression period.

11. The physiological signal measurement device according to claim 1, further comprising:
a notification unit configured to, in a case where the information processor determines that the change in the detected light intensity is inappropriate, perform at least one notification of a notification of a possibility that the compression is insufficient, a notification of prompting re-measurement by increasing an inflation target value, and a notification of prompting the re-measurement by increasing a compression time.

12. The physiological signal measurement device according to claim 1, wherein
the compression period is formed by automatically performing compression by an automatic compression device.

13. The physiological signal measurement device according to claim 1, wherein
in a case where the information processor determines that the change in the detected light intensity is inappropriate, one of an inflation target value and a compression time is increased and the physiological tissue is compressed, so that re-measurement is automatically performed.

14. The physiological signal measurement device according to claim 8, further comprising:
a notification unit configured to perform, in a case where the information processor determines that the change in the detected light intensity is inappropriate, at least one of a notification indicating a possibility that at least one of a position relation between the light emitter and the light detector and a compression direction is inappropriate and a notification prompting correction on positions of the light emitter or the light detector or a compression direction.

15. A physiological signal measurement system, comprising:
a physiological signal obtainer including a support tool configured to apply and release compression to a physiological tissue; and
the physiological signal measurement device according to claim 1.

16. A physiological signal measurement method comprising:
a capillary refilling time calculation procedure of calculating, based on change in a detected light intensity that is detected by a light detector configured to detect light emitted from a light emitter and transmitted through a physiological tissue, a capillary refilling time after a compression period in which the physiological tissue is compressed; and
a calculation validity determination step of using the detected light intensity to determine whether the change in the detected light intensity is appropriate for calculating the capillary refilling time.

17. A non-transitory computer readable storage medium storing a physiological signal measurement program for causing a computer to implement the following procedures:
a capillary refilling time calculation procedure of calculating, based on change in a detected light intensity that is detected by a light detector configured to detect light emitted from a light emitter and transmitted through a physiological tissue, a capillary refilling time after a compression period in which the physiological tissue is compressed; and
a calculation validity determination step of using the detected light intensity to determine whether the change in the detected light intensity is appropriate for calculating the capillary refilling time.
